(19) United States
(12) Patent Application Publication
(10) Pub. No.: US 2026/0069813 A1
(43) Pub. Date: Mar. 12, 2026

(54) GLASS-TYPE OXYGEN TUBE SUPPORTING DEVICE MADE OF SOFT RUBBER

(71) Applicant: Yuhua Jiang, Shaoyang (CN)

(72) Inventor: Yuhua Jiang, Shaoyang (CN)

(21) Appl. No.: 18/826,350

(22) Filed: Sep. 6, 2024

(51) Int. Cl. A61M 16/06; A61M 16/08; A61M 25/02; G02C 11/00

(52) U.S. Cl. CPC A61M 16/0683; A61M 16/0672; A61M 16/0875; G02C 11/00; A61M 2025/0206; A61M 2025/028

(57) ABSTRACT

A glass-type oxygen tube supporting device made of soft rubber comprises a hanging module, a base, a hanging hook, a structural connecting post, a connecting post insertion hole and an eyeglass temple fixing slit. The traditional nasal oxygen tube can cause discomfort and pain to ears after long-term use. Glasses are used as fulcrums, and a base made of a flexible rubber material is designed, which can adapt to different sizes and types of eyeglass temples. A C-shaped hook is arranged above the base to fix the nasal oxygen tube, which avoids the tube from sliding, and reduces the burden on the ears. The device is designed as a two-part structure. The base and hanging module can be integrated or movable as needed to adapt to the movement of the user's head. The base of the movable structure is capable of rotating by 360 degrees.

### TECHNICAL FIELD

The present disclosure relates to the technical field of medical auxiliary equipment and spectacle accessories, in particular to a glass-type oxygen tube supporting device made of soft rubber.

### BACKGROUND

Nasal oxygen tube is widely used in patients who need continuous oxygen supply, especially the elderly and long-term bedridden patients. However, the traditional nasal oxygen tube is usually fixed by hanging it on the ear, which can easily lead to ear discomfort, tenderness and skin wear during long-term use. Especially for the elderly users, because the skin is sensitive and fragile, the long-term bearing of the weight and friction of the nasal oxygen tube will aggravate the discomfort.
1. The existing oxygen tube structure will cause pressure burden and friction to the ear and its skin: pressure concentration: the ear, as the only fulcrum, bears all the weight of the oxygen tube, resulting in excessive local pressure. Long-term use will cause ear pain, and may even lead to indentation in the ear. Skin friction: because the oxygen tube is in direct contact with the skin of the ear, when the user's head moves, the tube will slide on the ear and generate friction. This kind of constant friction can easily lead to skin wear and discomfort, and may cause skin inflammation in severe cases. Poor adaptability: there are differences in the shape and sensitivity of different users'ears, so the existing design is difficult to meet the needs of all users, especially for the elderly with fragile skin and patients who wear oxygen tubes for a long time, which is easy to aggravate discomfort. Interference with glasses: many users of nasal oxygen tube need to wear glasses at the same time, and the existing design is easy to interfere with the glasses feet, which affects the stability and comfort of wearing, so there is an urgent need for an oxygen tube supporting device for glasses that can reduce the pressure and friction of ears.
2. The existing hanging devices are not universal: incompatible sizes: the existing hanging devices are usually designed for specific sizes and shapes, and it is difficult to adapt to different kinds and sizes of articles. For example, in the case of different shapes and sizes of eyeglass temples and ears, it is difficult for hanging devices to provide consistent fixing effect, which affects their wide application. Single fixing method: many hanging devices adopt fixed and non-adjustable design, which makes them unable to meet the needs of different users. This single fixed way is rigid and inflexible in the face of various usage scenarios and requirements. Limited users: due to the design limitations, the existing hanging devices can only meet the needs of specific people, and cannot be widely used for users of different ages or physical conditions, especially for users with special needs, such as the elderly or long-term patients, with poor experience. Complicated installation: some hanging devices are complicated in design, complicated in installation or adjustment, and inconvenient for users to use. This problem is particularly obvious in the scene that needs frequent use or adjustment, which reduces the user's willingness to use, so there is an urgent need for an oxygen tube supporting device for glasses that can adapt to various eyeglass temple structures.
3. The existing oxygen tubes have poor stability, and may fall off: the fixation is not firm: many oxygen tubes are designed to be fixed by simply hanging the ear, and the oxygen tubes are prone to displacement during wearing due to the insufficient shape and friction of the ear itself, which leads to unstable fixation, especially when the user moves his head. Easy to slip off: due to the lack of effective support and multi-point fixation, the oxygen tube may gradually slip off after long-term use, especially when sweating or the skin becomes greasy, which will affect the use effect. Inability to adapt to dynamic changes: the user's head movement and posture changes often change the direction and size of the oxygen tube, and the existing design is difficult to adapt to these dynamic changes, which easily leads to the oxygen tube loosening or falling off. User interference: due to unstable wearing, users may need to adjust the position of oxygen tubes frequently in their daily activities, which increases the inconvenience of use, and even may interrupt the oxygen supply in some cases, which may cause potential risks to health. Therefore, an oxygen tube supporting device for glasses that can improve the stability of oxygen tubes is urgently needed.

### SUMMARY

The main purpose of the present disclosure is to provide a glass-type oxygen tube supporting device made of soft rubber, which can effectively solve the problems in the background technology.

In order to achieve the above purpose, the technical solution adopted by the present disclosure is as follows:

A glass-type oxygen tube supporting device made of soft rubber includes a hanging module and a base, wherein a lower end of the hanging module is connected with the base, and an upper end of the hanging module is provided with a hanging hook for hanging an oxygen tube; the hanging hook is flexible in structure and is capable of supporting and fixing the oxygen tube; the lower end of the hanging module is provided with a structural connecting post for connecting with the base, and a post structure of the structural connecting post is convenient to be firmly installed on the base; a connecting post insertion hole is provided in a middle position of the base, and the connecting post insertion hole is used for accurately butting with the structural connecting post; a side surface of the base is designed with an eyeglass temple fixing slit for fixing the base on an eyeglass temple to ensure the stability of the device.

Further, the hanging hook is of a C-shaped structure, which is designed to quickly fix the oxygen tube, and the base is capable of rotating by 360 degrees along the hanging module, so that the oxygen tube has greater flexibility and comfort during use.

Further, the eyeglass temple fixing slit is structure with two parallel seams, and the eyeglass temple fixing slit is designed to firmly fix the eyeglass temples therein; by inserting the eyeglass temples into the slit, the slit structure provides support at both sides of the eyeglass temples to form two stable fixing fulcrums, thus preventing the device from sliding or shifting during use.

Further, the base is made of flexible rubber, and the use of flexible rubber can effectively improve the comfort of the device, adapt to the shapes of different eyeglass temples, and have a certain anti-skid effect, so that the supporting device can be more firmly attached to glasses.

Further, a cross-shaped deformation hole position is designed at the edge of the connecting post insertion hole, and the connecting post insertion hole can generate appropriate deformation when the structural connecting post is inserted, so as to better fix the structural connecting post; a dimension between the connecting post insertion hole and the structural connecting post is designed such that a diameter of the front end of the structural connecting post is slightly larger than that of a base hole, which ensures the stability of connection and makes the installation and disassembly more convenient.

Another structural arrangement of the glass-type oxygen tube supporting device made of soft rubber includes a hanging module, a base, a hanging hook, a structural connecting post, a connecting post insertion hole and an eyeglass temple fixing slit, wherein the hanging module, the base and the hanging hook are made into an integrated structure through an integrated molding process without separate movable parts; the hanging hook is directly fixed on an upper part of the hanging module, the base is directly fixed on a lower part of the hanging module, and the eyeglass temple fixing slit at the side of the base is consistent with the above structural arrangement.

Further, the structural connecting post at the lower end of the hanging module and the connecting post insertion hole in the middle of the base are in a same position as an original structure, but the structure has no function.

Further, the hanging module, the base and the hanging hook are in a structure made by an integrated molding process to improve the connection stability of the device.

Compared with the prior art, the present disclosure has the following beneficial effects:
1. The present disclosure aims at avoiding the pressure and friction caused by the oxygen tube directly hanging on the ear by fixing the oxygen tube on the eyeglass temple, and fundamentally solving the problem of ear pain: relieving the ear pressure: dispersing the weight of the oxygen tube by using the eyeglass temple as the fulcrum, avoiding the excessive pressure borne by the ear as the sole fulcrum, and obviously alleviating the discomfort of the ear; reducing skin friction: the oxygen tube is no longer in direct contact with the ear skin, which reduces the friction caused by the sliding of the tube, effectively prevents skin wear and inflammation, and improves the wearing comfort; increasing the wearing stability: the oxygen tube is fixed on the eyeglass temples, and make use of the multi-point stress characteristics of glasses to make the oxygen tube more stable and not easy to slide down, thus enhancing the reliability and safety during use; compatibility and ease of use: flexible rubber material is used in the design, which can adapt to different sizes and types of eyeglass temples, ensure the wide applicability of the device, and maintain the simplicity and flexibility of operation.
2. The present disclosure is made of flexible rubber, which is flexible in design, and is suitable for different sizes and types of eyeglass temples. At the same time, the device is capable of rotating by 360 degrees, providing greater flexibility, adapting to the movement change of the user's head, and significantly increasing the universality of the device: wide compatibility: the flexible rubber material enables the device to adapt to different sizes and types of eyeglass temples, and can be stably fixed regardless of whether it is a thin metal eyeglass temple or a wide plastic eyeglass temple. This design significantly improves the universality of the device and is suitable for various user needs. Retractable design: the retractility of the device not only ensures the close cooperation with different eyeglass temples, but also facilitates users to make fine adjustments according to their personal wearing habits, thus enhancing the comfort and convenience of use. 360-degree rotation function: the hook part of the device is designed to rotate by 360 degrees, which can flexibly adapt to the movement of the user's head. When the user turns his head, lowers his head or does other actions, the oxygen tube will not generate excessive stress or displacement, thus maintaining stability and improving the use experience. Strong adaptability: due to its flexible design, the device can adapt to a variety of use scenarios and user behaviors, and can provide a stable and comfortable wearing experience whether in daily activities or in a specific medical environment.
3. The present disclosure stably supports the oxygen tube by setting the fulcrum on the glasses, which reduces the possible sliding of the oxygen tube in the use process: stable support: the eyeglass temples are used as the fulcrum, which provides a stable fixed position for the oxygen tube and avoids the instability caused by the oxygen tube directly hanging on the ear. This can prevent the oxygen tube from sliding due to the shape or movement of the ear, and ensure that the tube is always kept in the best position. Reducing sliding: the traditional nasal oxygen tube is easy to slide due to the movement of the head during wearing, but the sliding risk of the oxygen tube is significantly reduced by fixing the glasses fulcrum, thus avoiding discomfort and ear pain caused by sliding. Improving comfort: the stable fulcrum design not only ensures the stability of the oxygen tube, but also reduces the inconvenience caused by frequent adjustment of the tube position, so that users can wear the oxygen tube more comfortably in daily activities. Enhancing safety: the design of reducing sliding also reduces the risk of oxygen tube falling off, especially when users are engaged in activities, this stable support significantly improves the safety and reliability of the device.

### BRIEF DESCRIPTION OF DRAWINGS

FIG. 1 is a schematic diagram of the overall structure of the present disclosure;

FIG. 2 is a schematic diagram of the hook structure of the present disclosure;

FIG. 3 is a schematic diagram of the base structure of the present disclosure;

FIG. 4 is the second schematic diagram of the base structure of the present disclosure;

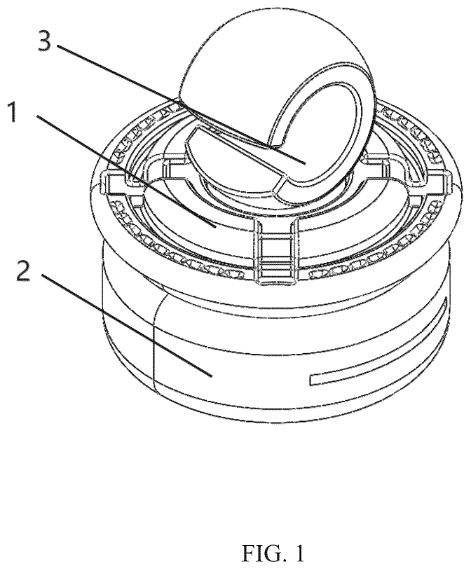

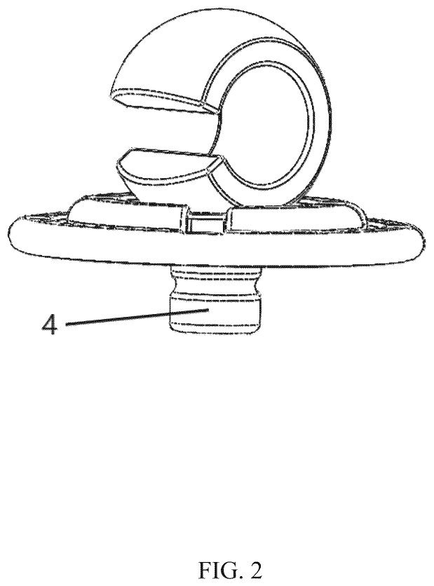

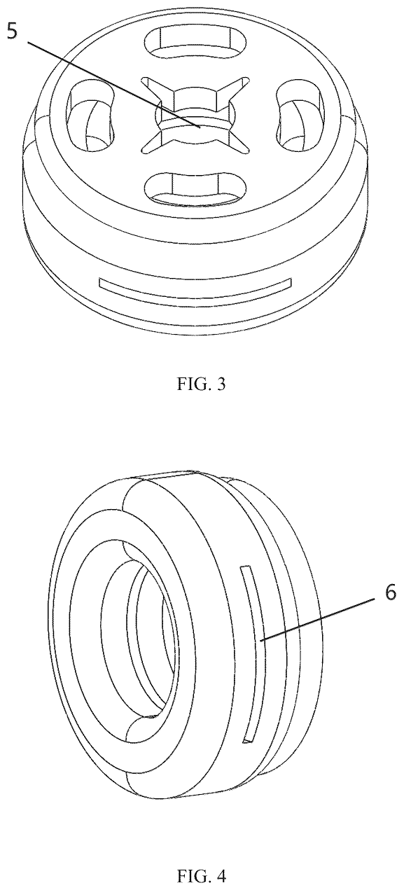

Reference signs: 1, hanging module; 2, Base; 3, Hanging hook; 4, Structural connecting post; 5, Connecting post insertion hole; 6, Eyeglass temple fixing slit.

### DETAILED DESCRIPTION OF THE EMBODIMENTS

In order to make the technical means, creative features, goals and effects of the present disclosure easy to understand, the present disclosure will be further elaborated with reference to specific examples.

In the description of the present disclosure, it should be noted that the azimuth or positional relationship indicated by the terms “upper”, “lower”, “inner”, “front end”, “rear end”, “one end” and “the other end” is based on the azimuth or positional relationship shown in the attached drawings, and is only for the convenience of describing the present disclosure and simplifying the description, and does not indicate or imply that the devices or elements referred to must have a specific orientation or a specific position. In addition, the terms “first” and “second” are only used for descriptive purposes and cannot be understood as indicating or implying relative importance.

In the description of the present disclosure, it should be noted that unless otherwise specified and limited, the terms “installation” and “connection” should be broadly understood, for example “connection” may an be a fixed connection, a detachable connection or an integrated connection; it may be a mechanical connection or an electrical connection; it may be direct connection, or indirect connection through an intermediate medium, and may be connection inside two elements. For those skilled in the art, the specific meanings of the above terms in the present disclosure can be understood in specific situations.

### EXAMPLES

The present disclosure provides a technical solution:

A glass-type oxygen tube supporting device made of soft rubber includes a hanging module 1 and a base 2, wherein a lower end of the hanging module 1 is connected with the base 2, and an upper end of the hanging module 1 is provided with a hanging hook 3 for hanging an oxygen tube; the hanging hook 3 is flexible in structure and is capable of supporting and fixing the oxygen tube; the lower end of the hanging module 1 is provided with a structural connecting post 4 for connecting with the base 2, and a post structure of the structural connecting post 4 is convenient to be firmly installed on the base 2; a connecting post insertion hole 5 is provided in a middle position of the base 2, and the connecting post insertion hole 5 is used for accurately butting with the structural connecting post 4; a side surface of the base 2 is designed with an eyeglass temple fixing slit 6 for fixing the base on an eyeglass temple to ensure the stability of the device.

The hanging hook 3 is of a C-shaped structure, which is designed to quickly fix the oxygen tube, and the base 1 is capable of rotating by 360 degrees along the hanging module 2, so that the oxygen tube has greater flexibility and comfort during use.

The eyeglass temple fixing slit 6 is structure with two parallel seams, and the eyeglass temple fixing slit 6 is designed to firmly fix the eyeglass temples therein; by inserting the eyeglass temples into the slit, the slit structure provides support at both sides of the eyeglass temples to form two stable fixing fulcrums, thus preventing the device from sliding or shifting during use.

The base 2 is made of flexible rubber, and the use of flexible rubber can effectively improve the comfort of the device, adapt to the shapes of different eyeglass temples, and have a certain anti-skid effect, so that the supporting device can be more firmly attached to glasses.

A cross-shaped deformation hole position is designed at the edge of the connecting post insertion hole 5, and the connecting post insertion hole 5 can generate appropriate deformation when the structural connecting post 4 is inserted, so as to better fix the structural connecting post; a dimension between the connecting post insertion hole 5 and the structural connecting post 4 is designed such that a diameter of the front end of the structural connecting post is slightly larger than that of a base hole, which ensures the stability of connection and makes the installation and disassembly more convenient.

Another structural arrangement includes a hanging module 1, a base 2, a hanging hook 3, a structural connecting post 4, a connecting post insertion hole 5 and an eyeglass temple fixing slit 6, wherein the hanging module 1, the base 2 and the hanging hook 3 are made into an integrated structure through an integrated molding process without separate movable parts; the hanging hook 3 is directly fixed on an upper part of the hanging module 1, the base 2 is directly fixed on a lower part of the hanging module 1, and the eyeglass temple fixing slit 6 at the side of the base 2 is consistent with the above structural arrangement.

The structural connecting post 4 at the lower end of the hanging module 1 and the connecting post insertion hole 5 in the middle of the base 2 are in a same position as an original structure, but the structure has no function.

The hanging module 1, the base 2 and the hanging hook 3 are in a structure made by an integrated molding process to improve the connection stability of the device.

The functions of each component of the present disclosure are as follows:

The present disclosure provides a glass-type oxygen tube supporting device made of soft rubber, which includes a hanging module and a base. The lower end of the hanging module is firmly connected with the base, and the upper end is provided with a hanging hook for supporting and fixing the oxygen tube. The hanging hook adopts C-shaped structure, which is flexible in design, and can easily and quickly hang the oxygen tube and ensure that it remains stable during use. The lower end of the hanging module is provided with a connecting post with a post structure, and the connecting post is accurately butted with the connecting post insertion hole in the middle of the base, so as to ensure the stable connection between the hanging module and the base.

When in use, the user hangs the oxygen tube on the hanging hook, and the design of the C-shaped hook can quickly fix the oxygen tube and keep it flexible and comfortable during use. The hanging hook and the hanging module are made into an integral structure through an integrated molding process, without any independent movable parts. The design between the hanging module and the hook ensures that there are no extra loose parts when in use, thus increasing the durability and stability of the device.

Through the design, the structural connecting post is capable of rotating horizontally in the connecting post insertion hole of the base, providing a 360-degree rotation function. This rotation function enables the oxygen tube to remain stable when the eyeglass temples are worn at different angles, and the wearing experience will not be affected by the angle change. No matter how the user's head rotates, the position of the oxygen tube can be flexibly adjusted to ensure comfort.

The side of the base is designed with two parallel slits for firmly fixing the device on the eyeglass temples. After the eyeglass temples are inserted into the slits, the two sides of the slits provide stable support points, effectively preventing the device from sliding or shifting during use. In addition, the base is made of flexible rubber, which can adapt to different specifications of eyeglass temples and provide good anti-skid effect, ensuring that the supporting device is firmly attached to the glasses.

During the installation and disassembly of the device, a cross-shaped deformation hole position is designed at the edge of the insertion hole of the connecting post to ensure that the connecting post can be properly deformed when inserted, so as to better fix the post. This design not only enhances the stability of the device, but also ensures the convenience of connection and disassembly.

A second structural embodiment relates to a glass-type oxygen tube supporting device made of soft rubber, which specifically includes a hanging module, a base, a hanging hook, a structural connecting post, a connecting post insertion hole and a fixing slit for eyeglass temples. The following is a detailed description of the device and its use process:

Structure of the device: hanging module: the hanging module, base and hanging hook of this embodiment are made into an integrated structure through an integrated molding process, and there is no separate movable part. The hanging hook is directly fixed on the upper part of the hanging module to ensure that the oxygen tube can be stably hung on the hook, and the lower part of the hanging module is fixed on the base to form an integral structure.

Base: the design of the base includes the eyeglass temple fixing slit, and the side structure is consistent with the integrated structure of the hanging module and the hanging hook. This design enables the base to be stably attached to the eyeglass temples.

Structural connecting post and connecting post insertion hole: the positions of the structural connecting post at the lower end of the hanging module and the connecting post insertion hole in the middle of the base are the same as those of the original structure, but these structures have no practical function, and are only used to increase the design consistency and overall beauty.

Usage: installation: the user aligns the eyeglass temple fixing slit of the base with the eyeglass temple for insertion. Because the base, the hanging module and the hanging hook are made by integrated molding process, the stability of the whole structure is improved. The design of the slit structure can firmly fix the base and prevent it from sliding or shifting.

Hanging oxygen tube: the user hangs the oxygen tube on the hanging hook. Due to the integrated design of hanging hook and hanging module, it can provide stable support for oxygen tube. The user does not need to make additional adjustment or fixing operation, and the structural design of the hook ensures the stable hanging of the oxygen tube.

Adjustment and use: due to the overall structural design of the base and hanging module, the device can provide good stability during use. Users do not need to make frequent adjustments during wearing, and the device can automatically adapt to the shape of glasses and provide comfortable support in actual use.

The glass-type oxygen tube supporting device combines the hanging module, the base and the hanging hook through an integrated molding process, and realizes a simple and stable design. The design of the eyeglass temple fixing silt and hanging hook of the base ensures the stable hanging of the oxygen tube and the firm attachment of the device, thus improving the wearing comfort and practicality.

On the whole, the supporting device provided by this embodiment is not only simple in structure and convenient to use, but also can provide a long-term, stable and comfortable oxygen tube supporting experience for the wearer.

In order to verify the advantages of the device, the following experimental procedures and corresponding data collection methods are designed to prove the effects of the device in reducing ear pressure, reducing skin friction, increasing wearing stability, enhancing universality, reducing sliding and improving comfort.

### Experiment 1: Verification of the Effect of Reducing Ear Pressure and Skin Friction.

Experimental target: to verify the effect of the device in reducing ear pressure and skin friction.
Experiment design:
Experimental group: the glass-type oxygen tube supporting device according to the present disclosure is used, and the oxygen tube is fixed by the eyeglass temples.
Control group: the traditional ear-hanging oxygen tube fixation method was used.
Experimental steps:
Subjects selection: 20 patients who used oxygen tubes for a long time were selected to ensure that they all wore oxygen tubes for a long time.
Pressure test: by wearing two different oxygen tubes (the experimental group and the control group), the pressure of the ear was measured by using a pressure sensor.
Test location: the upper and lower parts of the ear, and the area contacting the oxygen tube.
Test time: each group wore it for 2 hours, and the pressure data every 30 minutes were recorded.
Skin friction test: during wearing two different oxygen tube fixation methods, the skin condition of the subjects at the ear contact area was recorded, including redness, friction marks and self-reported scores of discomfort.
The surface temperature sensor was used to measure the temperature change of skin surface as an indirect index of friction.
Data collection and analysis:
Pressure data: the pressure value at each time point was recorded, and the average pressure difference between the experimental group and the control group was calculated.
Skin friction data: according to the self-reported scores and temperature changes of the subjects, the differences in friction between the experimental group and the control group were evaluated.

Experimental results show that the ear pressure of the experimental group is significantly lower than that of the control group, and the discomfort and temperature increase caused by skin friction are also lower than that of the control group, which proves that the present disclosure effectively reduces the ear pressure and reduces the skin friction.

### Experiment 2: Verification of the Universality and Wearing Stability of the Device.

Experimental target: to verify the effect of the device in adapting to different sizes of eyeglass temples and increasing wearing stability.
Experimental design:
Experimental group: the glass-type oxygen tube supporting device of the present disclosure was used.
Control group: different sizes of eyeglass temples were used, but this device was not used, the oxygen tube was directly hanged on ears.
Experimental steps:
Subjects selection: 20 subjects wearing glasses of different sizes and types were selected, including glasses wearers with thin metal legs and wide plastic legs.
Compatibility test: whether the device was installed smoothly on different sizes of eyeglass temples was tested, and whether the device can be fixed stably was observed.
The size and material type of each subject's eyeglass temples were recorded.
Stability test: by simulating daily head movements (such as turning head and lowering head) and testing whether the oxygen tube was displaced or slipped during wearing.
A position sensor was used to monitor the displacement of the oxygen tube.
Test time: each action was repeated for 10 times, and the displacement data of oxygen tube after each action was recorded.
Data collection and analysis:
Compatibility data: the compatibility of each size of eyeglass temples was recorded and the success rate of compatibility was counted.
Stability data: the displacement distance after different actions was recorded, and the stability difference between the experimental group and the control group was analyzed.
Experimental results: the device in the experimental group showed a high degree of compatibility, which was suitable for different sizes and types of eyeglass temples, and kept the stability of the oxygen tube during the head movement, which proved that the device had wide universality and high stability.

### Experiment 3: Verification of the Fulcrum Stability and Sliding Reduction Effect of the Device.

Experimental target: to verify the effect of the device in reducing oxygen tube sliding and improving comfort.
Experimental design:
Experimental group: the glass-type oxygen tube supporting device of the present disclosure was used.
Control group: the traditional ear-hanging oxygen tube fixation method was used.
Experimental steps:
Subjects selection: 10 subjects using oxygen tubes were selected.
Sliding test: during wearing the oxygen tube, the sliding frequency and amplitude of the oxygen tube was recorded.
Test environment: subjects wore oxygen tubes under the condition of simulating daily activities, such as walking, lowering their heads and turning their heads.
Test time: each group was tested for 2 hours, and the times of sliding and the displacement distance were recorded.
Comfort test: subjects filled in the comfort questionnaire after each group of experiments, and scored the discomfort caused by sliding and the overall wearing comfort.
Data collection and analysis:
Sliding data: the frequency and amplitude of sliding was recorded, and the sliding reduction rate of the experimental group and the control group was calculated.
Comfort score: the self-rated comfort score of the subjects was analyzed and the comfort differences between the two groups were compared.
Experimental results: the experimental group showed a significant reduction in sliding, and the comfort score was significantly higher than that of the control group, which proved that the device reduced the sliding of the oxygen tube by stabilizing the fulcrum and improved the overall wearing comfort.

Through these experimental procedures and data analysis, it can be strongly proved that the present disclosure does have the advantages of relieving ear pressure, reducing skin friction, increasing wearing stability, enhancing universality and reducing sliding in practical use.

According to the structural design of the present disclosure, the oxygen tube is fixed on the eyeglass temple, so that discomfort caused by the oxygen tube directly hanging on the ear is effectively avoided, and a more comfortable and stable wearing experience is provided, which is particularly suitable for users who need to wear the oxygen tube for a long time.

The basic principle, main features and advantages of the present disclosure have been shown and described above. It should be understood by those skilled in the art that the present disclosure is not limited by the above-mentioned examples, and what is described in the above-mentioned examples and descriptions only illustrates the principles of the present disclosure. Without departing from the spirit and scope of the present disclosure, there will be various changes and improvements in the present disclosure, which fall within the scope of the claimed disclosure. The scope of that present disclosure is define by the appended claim and their equivalents.

What is claimed is:

1. A glass-type oxygen tube supporting device made of soft rubber, comprising a hanging module (1) and a base (2), wherein a lower end of the hanging module (1) is connected with the base (2), and an upper end of the hanging module (1) is provided with a hanging hook (3) for hanging an oxygen tube; the hanging hook (3) is flexible in structure and is capable of supporting and fixing the oxygen tube; the lower end of the hanging module (1) is provided with a structural connecting post (4) for connecting with the base (2), and a post structure of the structural connecting post (4) is convenient to be firmly installed on the base (2); a connecting post insertion hole (5) is provided in a middle position of the base (2), and the connecting post insertion hole (5) is used for accurately butting with the structural connecting post (4); a side surface of the base (2) is designed with an eyeglass temple fixing slit (6) for fixing the base on an eyeglass temple to ensure the stability of the device.

2. The glass-type oxygen tube supporting device made of soft rubber according to claim 1, wherein the hanging hook (3) is of a C-shaped structure, which is designed to quickly fix the oxygen tube, and the base (1) is capable of rotating by 360 degrees along the hanging module (2), so that the oxygen tube has greater flexibility and comfort during use.

3. The glass-type oxygen tube supporting device made of soft rubber according to claim 1, wherein the eyeglass temple fixing slit (6) is structure with two parallel seams, and the eyeglass temple fixing slit (6) is designed to firmly fix the eyeglass temples therein; by inserting the eyeglass temples into the slit, the slit structure provides support at both sides of the eyeglass temples to form two stable fixing fulcrums, thus preventing the device from sliding or shifting during use.

4. The glass-type oxygen tube supporting device made of soft rubber according to claim 1, wherein the base (2) is made of flexible rubber, and the use of flexible rubber can effectively improve the comfort of the device, adapt to the shapes of different eyeglass temples, and have a certain anti-skid effect, so that the supporting device can be more firmly attached to glasses.

5. The glass-type oxygen tube supporting device made of soft rubber according to claim 1, wherein a cross-shaped deformation hole position is designed at the edge of the connecting post insertion hole (5), and the connecting post insertion hole (5) can generate appropriate deformation when the structural connecting post (4) is inserted, so as to better fix the structural connecting post; a dimension between the connecting post insertion hole (5) and the structural connecting post (4) is designed such that a diameter of the front end of the structural connecting post is slightly larger than that of a base hole, which ensures the stability of connection and makes the installation and disassembly more convenient.

6. A glass-type oxygen tube supporting device made of soft rubber, another structural arrangement of which comprises a hanging module (1), a base (2), a hanging hook (3), a structural connecting post (4), a connecting post insertion hole (5) and an eyeglass temple fixing slit (6), wherein the hanging module (1), the base (2) and the hanging hook (3) are made into an integrated structure through an integrated molding process without separate movable parts; the hanging hook (3) is directly fixed on an upper part of the hanging module (1), the base (2) is directly fixed on a lower part of the hanging module (1), and the eyeglass temple fixing slit (6) at the side of the base (2) is consistent with the above structural arrangement.

7. The glass-type oxygen tube supporting device made of soft rubber according to claim 6, wherein the structural connecting post (4) at the lower end of the hanging module (1) and the connecting post insertion hole (5) in the middle of the base (2) are in a same position as an original structure, but the structure has no function.

8. The glass-type oxygen tube supporting device made of soft rubber according to claim 6, wherein the hanging module (1), the base (2) and the hanging hook (3) are in a structure made by an integrated molding process to improve the connection stability of the device.
